(19) United States
(12) Patent Application Publication
(10) Pub. No.: US 2020/0324101 A1
(43) Pub. Date: Oct. 15, 2020

(54) ERGONOMIC CONNECTOR HUB FOR AN INFUSION SET

(71) Applicant: Eli Lilly and Company, Indianapolis, IN (US)

(72) Inventors: Daniel Morris HARTMANN, Arlington, MA (US); Sean Matthew PSZENNY, Cambridge, MA (US)

(21) Appl. No.: 16/094,576

(22) PCT Filed: Apr. 20, 2017
(86) PCT No.: PCT/US2017/028486

§ 371 Date: Oct. 18, 2018

Related U.S. Application Data
(60) Provisional application No. 62/326,153, filed on Apr. 22, 2016.

(51) Int. Cl. A61M 39/10

(52) U.S. Cl. CPC A61M 39/1055; A61M 2039/0036; A61M 2205/586; A61M 39/105

(57) ABSTRACT

The present disclosure relates to a connector hub adapted for use with an infusion set. The connector hub has a first connector and a second connector. The first connector communicates with a first fluid channel and has a first interface. The second connector communicates with a second fluid channel and has a second interface. Attaching the first and second connectors engages the first and second interfaces and provides fluid communication between the first and second fluid channels. Fluid flows between the medication reservoir and a cannula through the first and second fluid channels. At least one of the connectors may include a repositionable pivot member defining an interface.

### CROSS REFERENCE TO RELATED APPLICATIONS

This application claims priority of U.S. Provisional Patent Application Ser. No. 62/326,153 filed on Apr. 22, 2016 entitled ERGONOMIC CONNECTOR HUB FOR AN INFUSION SET, the entire disclosure of which is hereby incorporated by reference herein.

### BACKGROUND

The present disclosure relates to infusion sets for delivering a medication to a patient. Infusion sets typically include infusion line or tubing that leads from a medication delivery device, such as a pump, to a cannula inserted into the subcutaneous tissue of the patient. Such infusion sets are often used to inject insulin or other suitable medications.

Many infusion sets include a connector hub disposed between the medication pump and the cannula. Connector hubs include two mating parts that allow the pump to be connected and disconnected from the cannula without having to remove the cannula from the subcutaneous tissue. A conventional connector hub includes a female part having a septum and a male part having a needle that pierces the septum when the male and female parts are joined together.

In some infusion sets, the connection hub also functions as an insertion hub with the cannula being located at the same hub as the septum/needle assembly connection. In other infusion sets, the connector hub is separate from the insertion hub, and a length of tubing extends from the connector hub housing the septum/needle assembly to the insertion hub housing the cannula.

In both types of infusion sets, the connector hub typically attaches to the skin of the user and has a small size, low profile and rounded edges. This configuration may minimize discomfort to the user while reducing the likelihood of the connector hub snagging on clothing during wear. These same properties, i.e., small size, low profile and rounded edges, also make it difficult for users to grasp and manipulate the connector hub. For example, patients with large fingers, arthritis, or otherwise decreased manual dexterity may find it difficult and cumbersome to connect and disconnect the male and female portions of the connector hub.

### SUMMARY

The present disclosure provides an ergonomically enhanced connector hub for an infusion set that facilitates the manual manipulation of the connector hub during the connection and disconnection of the portions of the connector hub.

In accordance with a first aspect of the present disclosure, a connector hub adapted for use with an infusion set having a medication reservoir and a cannula to deliver medication to a patient. The connector hub includes a first connector in fluid communication with a first fluid channel. The first connector also has a first interface. The connector hub also includes a second connector in fluid communication with a second fluid channel wherein the second connector has a second interface. The first and second connectors are selectively attachable together wherein, when the first and second connectors are attached, the first and second interfaces are engaged and provide fluid communication between the first and second fluid channels. Detachment of the first and second connectors disengages the first and second interfaces and thereby disconnects the first and second fluid channels. Fluid flow between the medication reservoir and the cannula defines a fluid path that includes the first and second fluid channels. The second connector also has an adhesive layer and is adapted to be adhesively attached to the patient. One of the first and second connectors further includes a repositionable grip member, the grip member being moveable between a deployed position wherein the grip member projects outwardly from the one connector whereby the patient may grasp the grip member when connecting and disconnecting the first and second connectors and a low-profile storage position.

In some embodiments, the grip member is pivotally connected. The grip member may be disposed on the second connector. In some embodiments where the grip member is pivotally connected, the connector to which it is attached defines a recess and the grip member is at least partially disposed in the recess when the grip member is disposed in the storage position.

In some embodiments, the grip member is disposed on the second connector, the first interface comprises a piercing member in fluid communication with the first fluid channel, and the second interface comprises a septum controlling access to the second fluid channel. In some embodiments, when the first and second connectors are attached, the piercing member projects through the septum to provide fluid communication between the first and second fluid channels, and detachment of the first and second connectors removes the piercing member from the septum and thereby disconnects the first and second fluid channels. In such an embodiment, the piercing member may extend in a direction parallel with the adhesive layer when the first and second connectors are attached. The second fluid channel may also be adapted to be connected with a separate insertion hub housing the cannula.

In accordance with another aspect of the disclosure, a connector hub adapted for use with an infusion set having a medication reservoir and a cannula to deliver medication to a patient. The connector hub includes a first connector and a second connector. The first connector is in fluid communication with a first fluid channel and has a first interface. The second connector is in fluid communication with a second fluid channel and has a second interface. The first and second connectors are selectively attachable together wherein, when the first and second connectors are attached, the first and second interfaces are engaged and provide fluid communication between the first and second fluid channels. Detachment of the first and second connectors disengages the first and second interfaces and thereby disconnects the first and second fluid channels. Fluid flow between the medication reservoir and the cannula defines a fluid path including the first and second fluid channels. The second connector includes a base member and a pivot member. The base member has an adhesive layer adapted to be adhesively attached to the patient. The pivot member defines the second interface and is repositionable relative to the base member. The pivot member has a first position wherein the pivot member is disposed proximate the base member and a second position wherein the pivot member projects outwardly from the base member.

In some embodiments, the second fluid channel is adapted to be connected with a separate insertion hub housing the cannula. In other embodiments, the second fluid channel is in communication with the cannula and the cannula is mounted on the second connector.

The first interface may take the form of a piercing member in fluid communication with the first fluid channel and the second interface may take the form of a septum controlling access to the second fluid channel wherein, when the first and second connectors are attached, the piercing member projects through the septum to provide fluid communication between the first and second fluid channels, and detachment of the first and second connectors removes the piercing member from the septum and thereby disconnects the first and second fluid channels. In such embodiment, the connector hub may be configured such that, when the first and second connectors are attached together, the piercing member is disposed parallel with the adhesive layer in the first position and the piercing member is disposed at an angle to the adhesive layer in the second position.

In accordance with yet another aspect of the disclosure, a connector hub adapted for use with an infusion set having a medication reservoir and a cannula to deliver medication to a patient. The connector hub includes a first connector and a second connector. The first connector has a piercing member wherein the piercing member is in fluid communication with a first fluid channel. The second connector has a septum controlling access to a second fluid channel. The first and second connectors are selectively attachable together wherein, when the first and second connectors are attached, the piercing member projects through the septum to provide fluid communication between the first and second fluid channels, and wherein detachment of the first and second connectors removes the piercing member from the septum and thereby disconnects the first and second fluid channels. Fluid flow between the medication reservoir and the cannula defines a fluid path including the first and second fluid channels. The second connector has an adhesive layer and is adapted to be adhesively attached to the patient. The piercing member projects through the septum in a direction perpendicular to the adhesive layer when the first and second connectors are attached together.

In some embodiments, the second fluid channel is in communication with the cannula and the cannula is mounted on the second connector. In such an embodiment the cannula and the piercing member may extend in a same direction when the first and second connectors are attached together. In still additional embodiments, at least one of the first and second connectors may include a magnet wherein the magnet exerts a force resisting separation of the first and second connectors when the first and second connectors are attached together.

### BRIEF DESCRIPTION OF THE DRAWINGS

The features and advantages of the present disclosure will become more apparent to those skilled in the art upon consideration of the following detailed description taken in conjunction with the accompanying drawings, wherein:

FIG. 1 is a schematic view of an embodiment wherein the connector hub is also the insertion hub.

FIG. 2 is a schematic view of an embodiment wherein the connector hub is separate from the insertion hub.

FIG. 3 is a perspective view of an embodiment of a connector hub with first and second connectors detached.

FIG. 4 is a perspective view of the connector hub of FIG. 3 with the first and second connectors attached and a grip member in a deployed position.

FIG. 5 is a perspective view of the connector hub of FIG. 3 with the first and second connectors attached and the grip member in a storage position.

FIG. 6 is a schematic side view of the connector hub of FIG. 3 with the first and second connectors detached and the grip member in the storage position.

FIG. 7 is a perspective view of another embodiment of a connector hub with the first and second connectors detached.

FIG. 8 is a perspective view of the connector hub of FIG. 7 with the first and second connectors attached and a pivot member pivoted outwardly from a base member.

FIG. 9 is a perspective view of the connector hub of FIG. 7 with the first and second connectors attached and the pivot member positioned proximate the base member.

FIG. 10 is a perspective view of another embodiment of a connector hub with first and second connectors detached.

FIG. 11 is a perspective view of the connector hub of FIG. 10 with the first and second connectors attached and a pivot member pivoted outwardly from a base member.

FIG. 12 is a perspective view of the connector hub of FIG. 10 with the first and second connectors attached and the pivot member positioned proximate the base member.

FIG. 13 is a side view of another embodiment of a connector hub with first and second connectors attached.

FIG. 14 is a perspective view of the connector hub of FIG. 13 with the first and second connectors attached.

FIG. 15 is a schematic side view of a further embodiment of a connector hub with first and second connectors detached.

Corresponding reference characters indicate corresponding parts throughout the several views. The exemplifications set out herein illustrate exemplary embodiments of the invention, and such exemplifications are not intended to be exhaustive or to be construed as limiting the scope of the invention to the precise forms disclosed.

### DETAILED DESCRIPTION

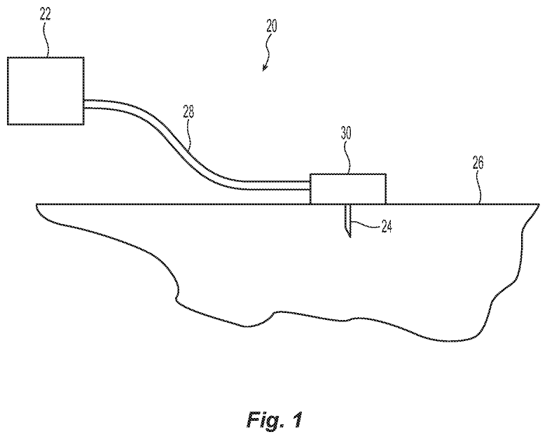

FIG. 1 schematically depicts an infusion set 20 having a cannula 24 to deliver medication from a medication reservoir 22 to a patient 26. Medication reservoir 22 may take the form of a pump operably coupled with a reservoir whereby the pump discharges the medication, such as insulin for example, which follows a fluid path 28 to cannula 24 where it is subcutaneously injected into patient 26. Infusion set 20 has a combined connection/insertion hub 30 which is attached to the skin of the patient. In the illustrative embodiment, flexible tubing (e.g., plastic) defines the fluid path 28 between reservoir 22 and hub 30.

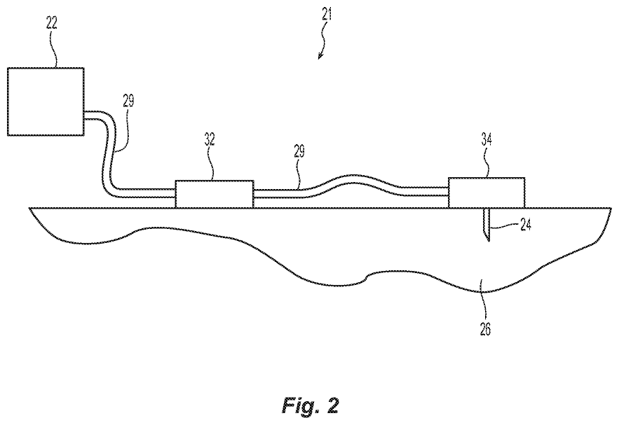

FIG. 2 schematically depicts an infusion set 21 having a connector hub 32 that is physically separate from an insertion hub 34. Infusion set 21 includes a fluid path 29 connected to medication reservoir 22 and hubs 32, 34 for delivering fluid medication to cannula 24 of hub 34. In this embodiment, flexible tubing (e.g., plastic) defines the fluid path 29 between reservoir 22 and hub 32 and between hub 32 and hub 34. In one embodiment, both hubs 32, 34 include an adhesive backing for attaching to the skin of the patient.

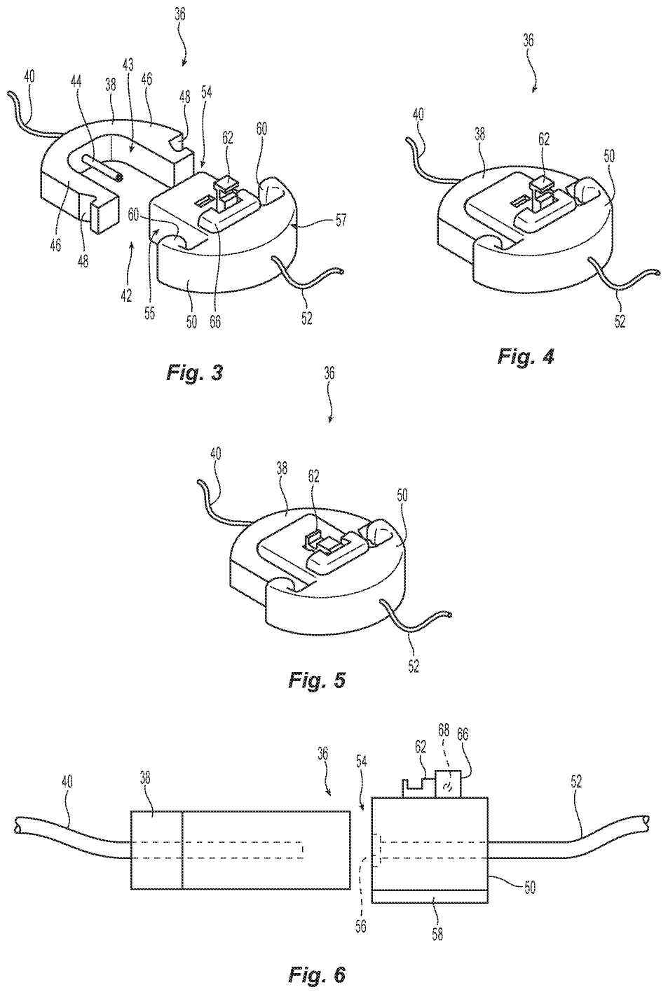

FIGS. 3-6 illustrate one example of a connector hub 36 that can be employed with infusion set 21. Connector hub 36 of FIGS. 3-6 includes a first connector 38 in fluid communication with a first fluid channel 40 and a second connector 50 in fluid communication with a second fluid channel 52. First connector 38 includes a first interface 42 which mates with a second interface 54 of second connector 50. First interface 42 includes a hollow piercing member 44, such as a needle or cannula, which is in fluid communication with the first fluid channel 40 extending through connector 38. In the illustrated embodiment, first fluid channel 40 is defined by flexible plastic tubing extending outside of the body of first connector 38 and also includes a segment within the connector 38 body. The portion of the first fluid channel 40 located within the first connector 38 may be defined by flexible plastic tubing or simply by a channel formed by the material used to form connector 38, such as a thermoplastic material, for example. First fluid channel 40 may be used to provide fluid communication between reservoir 22 and first connector 38.

First interface 42 of first connector 38 also includes a pair of flexible resilient attachment arms 46 which include recesses 48 illustratively formed in the outer surface of arms 46. Arms 46 engage with second connector 50 via the cooperation of recesses 48 and locking members 60 to secure the two connectors 38, 50 together as further discussed below. Arms 46 form a slot 43 in first interface 42 sized to receive a body portion 55 of second connector 50.

Second connector 50 of connector hub 36 is in fluid communication with a second fluid channel 52 and defines a second interface 54. Second connector 50 includes body portion 55 and an arc-shaped head portion 57. Similar to first fluid channel 40, second fluid channel 52 is defined by a flexible plastic tube positioned outside of the connector body and includes an internal portion extending through body and head portions 55, 57 of connector 50. The end of fluid channel 52 located at second interface 54 includes a septum 56 (e.g., rubber material) that controls access to the second fluid channel 52. In the illustrated embodiment of FIGS. 3-6, the second fluid channel 52 provides fluid communication to a separate insertion hub that houses a cannula. in an alternative embodiment, second connector 50 may be integrated with an insertion hub as a single part containing the cannula, as described herein.

The illustrated second connector 50 has an adhesive layer 58 (FIG. 6) and is adapted to be adhesively attached to the patient 26. In a preferred embodiment, the connector which is not directly coupled to the medication reservoir (i.e., second connector 50) is the sole connector of hub 36 that is adhesively secured to the patient. As such, the connector coupled to the medication reservoir (e.g., first connector 38) is removable and replaceable without removing the adhesive interface of the second connector 50 from the patient

When the first and second connectors 38, 50 are attached, piercing member 44 projects through septum 56 to provide fluid communication between the first and second fluid channels 40, 52. Fluid flow between the medication reservoir 22 and the cannula 24 defines a fluid path that includes the first and second fluid channels 40, 52. Detachment of the first and second connectors 38, 50 removes the piercing member 44 from the septum 56 and thereby disconnects the first and second fluid channels. For example, this allows for the attachment of a new medication reservoir or to temporarily detach the reservoir and associated pump from the body of the patient when taking a shower or performing some other task.

In the embodiment illustrated in FIGS. 3-6, piercing member 44 extends in a direction parallel with the adhesive layer 58 when the first and second connectors 38, 50 are attached.

Second connector 50 includes locking members 60 that engage recesses 48 on arms 46 to secure connectors 38, 50 when attached. To disengage the connectors 38, 50, arms 46 are pressed and flexed inwardly to disengage locking members 60 from recesses 48, and first connector 38 is withdrawn from second connector 50. To facilitate the patient's ability to grasp the second connector 50, a repositionable grip member 62 is disposed on second connector 50. Grip member 62 is illustratively positioned on a top surface opposite the adhesive patch 58 but may be positioned in other suitable locations on connector 50.

Although in the illustrated embodiment a grip member 62 is disposed on second connector 50, alternative embodiments may include a grip member 62 on both first and second connectors 38, 50 or just on first connector 38.

Grip member 62 is moveable between a deployed position (FIGS. 3, 4) and a low-profile storage position (FIG. 5). In the deployed position, the grip member 62 projects outwardly from connector 50 whereby the patient may readily grasp the grip member 62 when connecting and disconnecting the first and second connectors 38, 50.

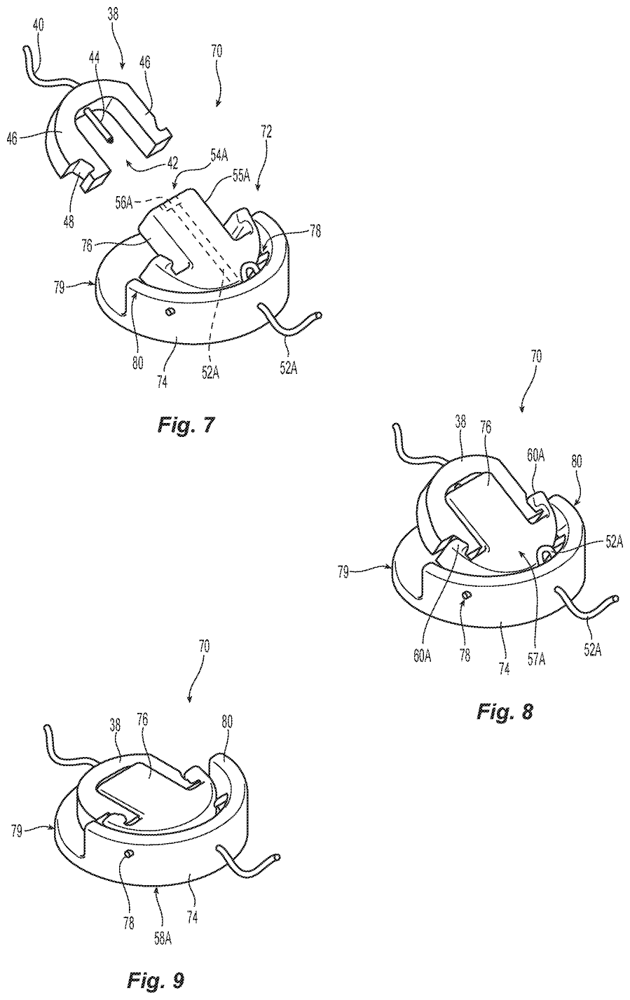

In the illustrated embodiment, grip member 62 is pivotally connected to a raised portion 66 of second connector 50 with a pin 68 which provides an axis on which grip member 62 pivots. Connector 50 also includes a recess 64 which receives at least a portion of grip member 62 when it is in the storage position. Recess 64 illustratively receives a tab portion on an end of grip member 62. The positioning of a portion of grip member 62 in recess 64 may engage via a friction fit or snap fit of grip member 62 with recess 64 to secure grip member 62 in the storage position until the user pulls grip member 62 up into the deployed position. In an alternative embodiment, body portion 55 of connector 50 includes a larger recessed area to receive a majority of grip member 62 in the deployed position. In this embodiment, a portion of grip member 62 extends above the plane formed by the top surface of body portion 55 to allow a user to grasp and move grip member 62 from the storage position to the deployed position. A grip member 62 may also be provided in the embodiments of FIGS. 7-15.

Another embodiment of a connector hub is illustrated in FIGS. 7-9. Connector hub 70 illustrated in FIGS. 7-9 is configured for use with an infusion set 21 as depicted in FIG. 2 wherein the connector hub is separate from the insertion hub.

The first connector 38 of hub 70 has the same design as the first connector of the hub 36 illustrated in FIGS. 3-6 and described herein. Second connector 72 includes a base member 74 and a pivot member 76. Pivot member 76 is similar in design to second connector 50 but, in the illustrated embodiment, does not include grip member 62, although a grip member 62 may be provided on second connector 72. Pivot member 76 includes a second interface 54A having a same design as interface 54 and that mates with first interface 42. Second interface 54A includes a septum 561. which controls access to second fluid channel 52A.

Similar to the embodiment of FIGS. 3-6, connector hub 70 of FIGS. 7-9 has first and second connectors 38, 72 which are selectively attachable together wherein, when the first and second connectors 38, 72 are attached, the first and second interfaces 42, 54A are engaged and provide fluid communication between the first and second fluid channels 40, 52A. In the illustrated embodiment, first interface 42 includes a piercing member 44 that pierces septum 56A of second interface 54A to provide the fluid communication. Pivot member 76 includes locking members 60A that engage recesses 48 on arms 46 of first connector 38 to secure the connectors 38, 72 together when the first and second interfaces 42, 54A are engaged.

Detachment of the first and second connectors 38, 72 disengages the first and second interfaces and thereby disconnects the first and second fluid channels 40, 52A. Arms 46 are configured to bias inwardly to allow first connector 38 to be removed from second connector 72. Fluid flow between the medication reservoir 22 and the cannula 24 defines a fluid path that includes the first and second fluid channels 40, 52A.

As mentioned above, the illustrated embodiment shown in FIGS. 7-9 has a second fluid channel 52A that is adapted to be connected with a separate insertion hub that houses a cannula. Alternatively, fluid channel 52A could extend to a cannula mounted on base member 74 to provide a combined connection and insertion hub as depicted in FIG. 1.

Base member 74 includes a disc-shaped lower portion 79 and a raised shoulder portion 80 extending along a portion of the outer perimeter of lower portion 79. Shoulder portion 80 extends outwardly from lower portion 79 in a direction opposite the adhesive layer. Illustratively, shoulder 80 extends in an arc shape along about half of the outer perimeter of lower portion 79 and to a height approximately the same as the height of pivot member 76. The arc shape of raised shoulder 80 follows a similar contour to the arc shape of head portion 57A such that, in assembly, shoulder 80 receives head portion 57A while remaining spaced apart from head portion 57A. Base member 74 has an adhesive layer 58A fixed to a bottom surface of lower portion 79 and adapted to be adhesively attached to the patient.

Pivot member 76 is pivotally mounted to base member 74 with a pivot axle 78 that extends through head portion 57A of pivot member 76 and raised shoulder 80 of base member 74. Pivot member 76 is repositionable relative to the base member 74 to facilitate the attachment and detachment of first connector 38 with second connector 72. The pivot member 76 has a first, lowered or down position (shown in FIG. 9) wherein pivot member 76 is disposed proximate base member 74 and a second, raised position (FIGS. 7, 8) wherein pivot member 76 projects outwardly from base member 74 to provide greater access to second interface 54A by a user. Second fluid channel 52A is formed in part by a length of flexible tubing extending from pivot member 76 to base member 74 that allows for the pivotal motion of pivot member 76 relative to base member 74.

In the embodiment of FIGS. 7-9, first interface 42 takes the form of a piercing member 44 in fluid communication with the first fluid channel, and second interface 54A takes the form of a septum controlling access to the second fluid channel. Connector hub 70 is configured such that, when the first and second connectors are attached together, piercing member 44 is disposed parallel with adhesive layer 58A in the first position (FIG. 9) and piercing member 44 is disposed at an angle to adhesive layer 58A in the second position (FIGS. 7, 8).

The use of a pivot member 76 facilitates the user's ability to attach and detach the first and second connectors 38, 72. The pivoting configuration of pivot member 76 allows second interface 54 on the distal end of pivot member 76 to face upward and away from the adhesive layer 58A which will be attached the patient's body. With the pivot member 76 pivoted outward, as shown in FIGS. 7, 8, the user will find it more convenient to attach and detach the first and second connectors 38, 72 compared to a configuration wherein the first connector must be moved into engagement in a direction parallel and proximate the plane defined by adhesive layer 58A. The direction in which the connectors 38, 72 are moved into and out of engagement is at an angle relative to adhesive layer 58A, thus it is also at an angle relative to the patient's skin to which the adhesive layer 58A is attached. When engaging first connector 38 with second connector 72, this angle of attachment will result in at least some of the force exerted by first connector 38 will press second connector 72 into further engagement with the user's skin and only some of the engagement force will be a force acting parallel to adhesive layer 58A. Raised shoulder 80 and/or head portion 57A also provide a convenient location for the user to grasp second connector 72 when attaching and detaching the connectors.

Pivoting pivot member 76 upward as shown in FIGS. 7, 8 also provides greater clearance around second interface 54A. which will make it easier for the user to manipulate first connector 38 as the user presses first connector 38 into engagement with second connector 72.

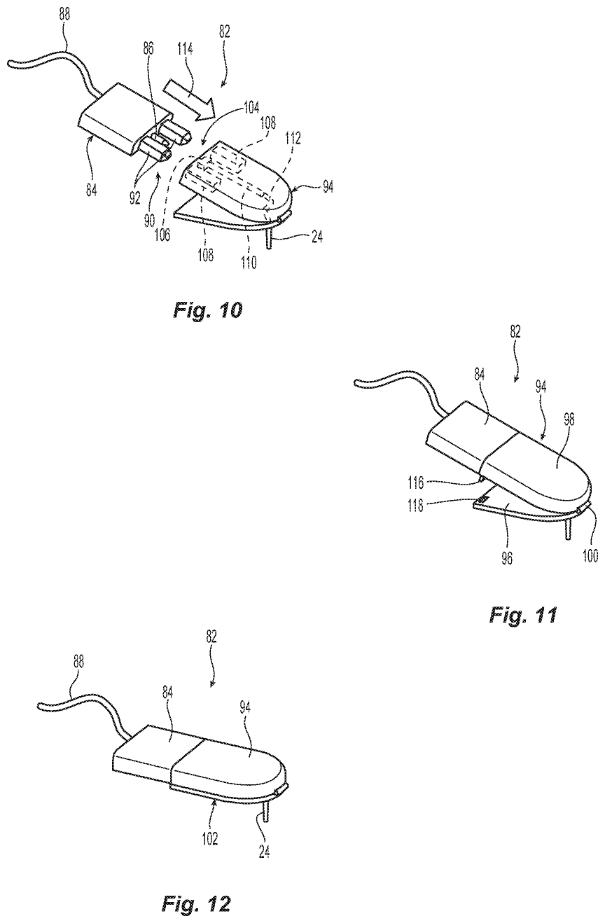

In a further embodiment, pivot member 76 and base member 74 are configured to snap-fit or interference fit in the lowered position, such as with a detent as described herein with respect to FIGS. 10-12, to resist movement of pivot member 76 relative to base member 74 when the hub 70 is in the lowered position while still permitting a user to disengage pivot member 76 from base member 74 with the application of a small force.

While the second fluid channel is adapted to be connected with a separate insertion hub housing the cannula in the embodiment of FIGS. 7-9, the embodiment of FIGS. 10-12 provides a combined connection and insertion hub wherein the second fluid channel is in communication with the cannula and the cannula is mounted on the second connector. FIG. 1 provides a schematic illustration of an insertion set 20 having such a combined connection and insertion hub.

FIGS. 10-12 illustrate a connector hub 82 that is also an insertion hub. Hub 82 includes a first connector 84 having a hollow piercing member 86 that is in fluid communication with flexible tubing 88 (e.g., plastic material) that functions as a first fluid channel in communication with a medication reservoir. First interface 90 of first connector 84 includes a pair of guide members 92 extending parallel to and on either side of piercing member 86. Second connector 94 includes a base member 96 and a pivot member 98 which are coupled together with a hinge 100. Hinge 100 may include a hinged bracket, a living hinge, or other suitable hinge. Hinge 100 allows pivot member 98 to move between a first, lowered position proximate base member 96 (FIG. 12) and a second, raised position spaced apart from base member 96 (FIGS. 10, 11).

An adhesive layer 102 is provided on a bottom surface of base member 96 for attaching hub 82 to a patient. Cannula 24 is also mounted on base member 96 and is inserted into the patient when attaching base member 96 on the patient. Pivot member 98 defines a second interface 104 which includes a septum 106 and a pair of guide channels 108. When first and second interfaces are engaged, as shown in FIGS. 11, 12, piercing member 86 pierces septum 106 and places first fluid channel 88 in fluid communication with second fluid channel 110. A portion of second fluid channel 110 is defined by a length of flexible tubing 112 extending between pivot member 98 and base member 96 which accommodates the pivotal movement of pivot member 98 relative to base member 96.

As can be seen in FIGS. 10, 11, pivot member 98 is configured to pivot to make second interface 104 more accessible for attaching and detaching first connector 84. Arrow 114 indicates the direction in which first connector 84 is moved into engagement with second connector 94. After sliding guide members 92 into guide channels 108 and extending piercing member 86 thorough septum 106 (FIG. 11), pivot member 94 is repositioned to the lowered position so that it is proximate base member 96 (FIG. 12). Base member 96 and pivot member 98 illustratively have a same outer profile, although base member 96 alternatively may extend wider than pivot member 98, such as to provide a wider surface on which a user may exert a downward force to facilitate attaching and detaching connectors 84, 94.

Projections 116 on pivot member 98 engage recesses 118 on base member 96 and form a snap-fit or interference fit to act as detents which resist movement of pivot member 98 relative to base member 96 when hub 82 is in the position shown in FIG. 12 but still permit a user to disengage pivot member 98 from base member 96 with the application of a small force. Similarly, guide members 92 and guide channels 108 may include interlocking projections and recesses or similar features to retain first and second connectors 84, 94 in their attached position but still permit a user to non-destructively disengage the first and second connectors. These or various other securement features, such as patches of interlocking hooks and loops or magnetic closures, may be employed with hub 82 and/or the other hub embodiments described herein.

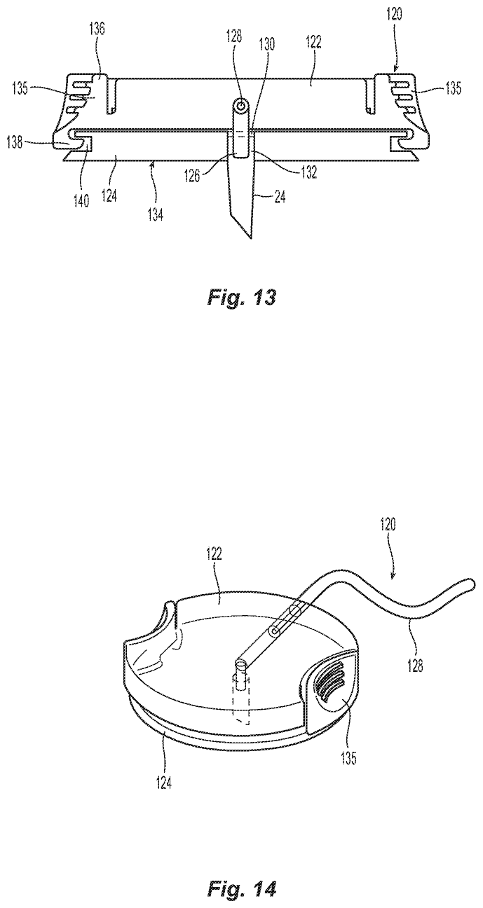

In the embodiment of FIGS. 13, 14, connector hub 120 is also an insertion hub and is suitable for use in an infusion system such as that schematically depicted in FIG. 1. Hub 120 includes a first connector 122 and a second connector 124. First connector 122 has a hollow piercing member 126 which is in fluid communication with a first fluid channel 128. Second connector 124 has a septum 130 controlling access to a second fluid channel 132. Second fluid channel 132 is in fluid communication with a cannula 24 mounted on second connector 126.

When the first and second connectors 122, 124 are attached, piercing member 126 projects through the septum 130 to provide fluid communication between the first and second fluid channels 128, 132. Detachment of the first and second connectors 122, 124 removes the piercing member 126 from the septum 130 and thereby disconnects the first and second fluid channels 128, 132. Fluid flow between the medication reservoir and the cannula 24 defines a fluid path that includes the first and second fluid channels 128, 132.

Second connector 124 has an adhesive layer 134 fixed to a bottom surface for adhesively attaching second connector 124 to the patient. The piercing member 126 projects through the septum 130 in a direction perpendicular to the adhesive layer 134 when the first and second connectors 122, 124 are attached together.

In the embodiment illustrated in FIGS. 13, 14, the second fluid channel 132 is in communication with the cannula 24 and the cannula 24 is mounted on the second connector 124 such that the cannula 24 and the piercing member 126 extend in a same or parallel direction when the first and second connectors 122, 124 are attached together. This arrangement results in first connector 122 being moved in a direction perpendicular to adhesive layer 134 as first connector 122 is attached to second connector 124 and piercing member 126 pierces septum 130. As a result, attachment of first connector 122 presses second connector 124 onto the patient's skin and helps to maintain hub 120 in place on the patient. Adhesive layer 134 is provided with enough strength to remain attached to the patient when first connector 122 is removed from second connector 124.

A pair of resilient latching members 135 are provided on first connector 122. for securing first connector 122 to second connector 124. Illustratively, latching members 135 are diametrically opposed to each other and are positioned on the outer perimeter of first connector 122, thereby allowing a user to grip and squeeze latching members 135 with one hand. Referring to FIG. 13, latch members 135 each have a lower hook portion 138 that engage with a corresponding recess 140 in base member 124 to secure the first and second connectors together. The upper part 136 of each latch 135 is configured to be biased inwardly upon application of a force by the patient which causes the lower hook portion 138 of latch 135 to move outwardly out of engagement with recess 140 during the attachment and removal of first connector 122 from second connector 124.

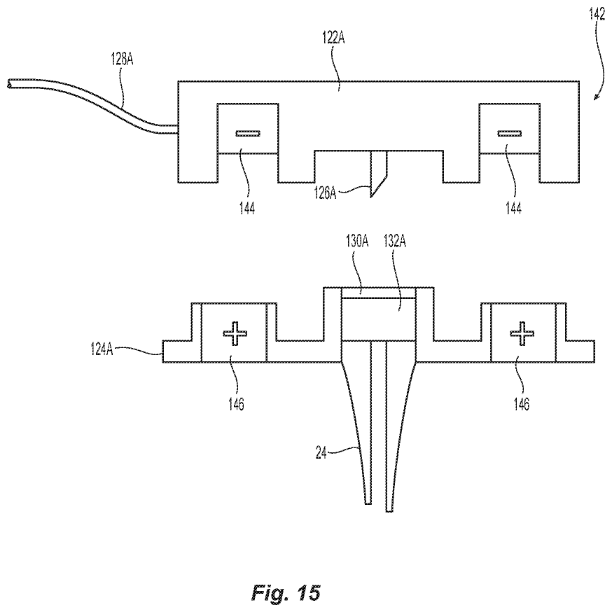

FIG. 15 schematically depicts a hub 142 having a design similar to hub 120 of FIGS. 13 and 14 but which uses a different mechanism to secure the first connector 122A to the second connector 124A. Like hub 120, hub 142 has a first connector 122A with a piercing member 126A and a first fluid channel 128A and a second connector 124A with a septum 130A and second fluid channel 132A wherein the piercing member 126A and cannula 24 extend in a same direction with each other when the connectors are secured together.

Instead of a latching mechanism, connector hub 142 uses a magnetic attachment to secure first and second connectors 122A, 124A. A first set of magnets 144 are located on first connector 122A on either side of piercing member 126A and a second set of magnets 146 are located on second connector 124A and axially aligned with magnets 144 of first connector 122A. Magnets 144, 146 are positioned so that they have surfaces of opposite polarity facing each other and thereby magnets 144 and magnets 146 will be attracted to each other and releasably secure connectors 122A, 124A together. In other words, the magnets will exert a force resisting separation of the first and second connectors 122A, 124A when the first and second connectors 122A, 124A are attached together. In one embodiment, the force required to overcome the magnetic attraction force is less than the force required to pull the adhesive of the second connector 124A away from the patient. In one embodiment, the second connector 124A includes a base that is wider than a width of the first connector 122A to provide a wider surface on which a user may exert a downward force to facilitate attaching and detaching connectors 122A, 124A.

Although the illustrated embodiment of FIG. 15 shows magnets located on each of the first and second connectors 122A, 124A, alternative embodiments could employ one or more magnets on one of the connectors and a ferrous metal component on the opposite connector to provide for a magnetic attachment system.

It is additionally noted that while the embodiments of FIGS. 13-15 are combined connector/insertion hubs, alternative embodiments could provide a flexible tube extending out the side or top of the connector hubs to connect with a separate insertion hub.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A connector hub adapted for use with an infusion set having a medication reservoir and a cannula to deliver medication to a patient, the connector hub comprising:
a first connector in fluid communication with a first fluid channel and having a first interface;
a second connector in fluid communication with a second fluid channel and having a second interface, the first and second connectors being selectively attachable together wherein, when the first and second connectors are attached, the first and second interfaces are engaged and provide fluid communication between the first and second fluid channels, and wherein detachment of the first and second connectors disengages the first and second interfaces and thereby disconnects the first and second fluid channels, and wherein fluid flow between the medication reservoir and the cannula defines a fluid path including the first and second fluid channels; and
wherein the second connector includes a base member and a pivot member, the base member having an adhesive layer adapted to be adhesively attached to the patient, and wherein the pivot member defines the second interface and is repositionable relative to the base member, the pivot member having a first position wherein the pivot member is disposed proximate the base member and a second position wherein the pivot member projects outwardly from the base member.

2. The connector hub of claim 1, wherein the first interface comprises a piercing member in fluid communication with the first fluid channel and the second interface comprises a septum controlling access to the second fluid channel wherein, when the first and second connectors are attached, the piercing member projects through the septum to provide fluid communication between the first and second fluid channels, and wherein detachment of the first and second connectors removes the piercing member from the septum and thereby disconnects the first and second fluid channels.

3. The connector hub of claim 2, wherein, when the first and second connectors are attached together, the piercing member is disposed parallel with the adhesive layer in the first position and the piercing member is disposed at an angle to the adhesive layer in the second position.

4. The connector hub of claim 1, wherein the base member includes a lower portion and a shoulder portion extending from the lower portion, and a pivot axis extends through the shoulder portion and the pivot member, the pivot member being moveable about the pivot axis between the first position and the second position.

5. The connector hub of claim 1, wherein the second fluid channel is in communication with the cannula and the cannula is mounted on the second connector.

6. A connector hub adapted for use with an infusion set having a medication reservoir and a cannula to deliver medication to a patient, the connector hub comprising:
a first connector having a piercing member, the piercing member in fluid communication with a first fluid channel;
a second connector having a septum controlling access to a second fluid channel, the first and second connectors being selectively attachable together wherein, when the first and second connectors are attached, the piercing member projects through the septum to provide fluid communication between the first and second fluid channels, and wherein detachment of the first and second connectors removes the piercing member from the septum and thereby disconnects the first and second fluid channels, and wherein fluid flow between the medication reservoir and the cannula defines a fluid path including the first and second fluid channels; and
wherein the second connector has an adhesive layer and is adapted to be adhesively attached to the patient, and wherein the piercing member projects through the septum in a direction perpendicular to the adhesive layer when the first and second connectors are attached together.

7. The connector hub of claim 6, wherein the second fluid channel is in communication with the cannula and the cannula is mounted on the second connector.

8. The connector hub of claim 7, wherein the cannula and the piercing member extend in a same direction when the first and second connectors are attached together.

9. The connector hub of claim 8, wherein at least one of the first and second connectors includes a magnet, and the magnet exerts a force resisting separation of the first and second connectors when the first and second connectors are attached together.

10. The connector hub of claim 8, wherein one of the first and second connectors includes a latch member configured to engage the other of the first and second connectors to secure the first and second connectors together while the piercing member projects through the septum, and the latch member is releasable to detach the first and second connectors.

11. A connector hub adapted for use with an infusion set having a cannula to deliver medication from a medication reservoir to a patient, the connector hub comprising:
a first connector in fluid communication with a first fluid channel and having a first interface;
a second connector in fluid communication with a second fluid channel and having a second interface, the first and second connectors being selectively attachable together wherein, when the first and second connectors are attached, the first and second interfaces are engaged and provide fluid communication between the first and second fluid channels, and wherein detachment of the first and second connectors disengages the first and second interfaces and thereby disconnects the first and second fluid channels, and wherein fluid flow between the medication reservoir and the cannula defines a fluid path including the first and second fluid channels;
wherein the second connector has an adhesive layer and is adapted to be adhesively attached to the patient; and
at least one of the first and second connectors further includes a repositionable grip member, the grip member being moveable between a deployed position wherein the grip member projects outwardly from the at least one connector whereby the patient may grasp the grip member when connecting and disconnecting the first and second connectors and a low-profile storage position.

12. The connector hub of claim 11, wherein the grip member is pivotally connected to the one connector.

13. The connector hub of claim 12, wherein the at least one connector defines a recess and the grip member is at least partially disposed in the recess when the grip member is disposed in the storage position.

14. The connector hub of claim 11, wherein the grip member is disposed on the second connector and wherein the first interface comprises a piercing member in fluid communication with the first fluid channel and the second interface comprises a septum controlling access to the second fluid channel wherein, when the first and second connectors are attached, the piercing member projects through the septum to provide fluid communication between the first and second fluid channels, and wherein detachment of the first and second connectors removes the piercing member from the septum and thereby disconnects the first and second fluid channels.

15. The connector hub of claim 14, wherein the piercing member extends in a direction parallel with the adhesive layer when the first and second connectors are attached, wherein the second connector defines a recess and the grip member is pivotally connected to the one connector, the grip member being at least partially disposed in the recess when the grip member is disposed in the storage position.
